(19) United States
(12) Patent Application Publication
(10) Pub. No.: US 2011/0262523 A1
(43) Pub. Date: Oct. 27, 2011

(54) MEDICAL ADHESIVE TAPE AND MEDICAL ARTICLES MADE OF THE SAME

(76) Inventors: Dunshen . Zhu, Shanghai (CN); ShiMing Zhou, Shanghai (CN); Ting Fan, Shanghai (CN); Grace L. Ho, Shanghai (CN); Dong Wu, Woodbury, MN (US)

(21) Appl. No.: 13/125,341

(22) PCT Filed: Oct. 20, 2009
(86) PCT No.: PCT/US09/61292

§ 371 Date: Jul. 11, 2011

(30) Foreign Application Priority Data
Oct. 22, 2008 (CN) 200810170076.3

(51) Int. Cl. A61K 9/70; C09J 7/04; A61P 17/02; A61F 13/02

(52) U.S. Cl. 424/448; 442/151

(57) ABSTRACT

The present invention provides a medical adhesive tape comprising a non-woven fabric substrate of a metallocene polypropylene copolymer; and a medical pressure-sensitive adhesive coated on at least one side of said substrate. A medical article comprising a medicament and the medical adhesive tape of the invention is also provided.

### FIELD OF THE INVENTION

The present invention relates to a medical adhesive tape and medical articles made of the same, and specifically, to a medical adhesive tape comprising a non-woven fabric as substrate material and medical articles made of the same, such as a medical dressing.

### BACKGROUND

In the field of and (consumer) care, non-woven fabric adhesive tapes are widely used because of their advantages of good performance, low price and convenient processability or the like. The raw materials of non-woven fabric adhesive tapes include rayon yarn, polyester, a mixture of rayon yarn/polyester and polypropylene or the like. For example, Chinese Patent CN2267786Y discloses an air permeable medical adhesive tape comprising a winding cylinder wherein one side of a non-woven fabric is provided with a medical base adhesive and the other side of the non-woven fabric is provided with an adhesive. A medical base adhesive is interposed between the adhesive and the non-woven fabric. The adhesive is a medical grade acrylic pressure-sensitive adhesive and the non-woven fabric is a conventional Ziegler-Natta polypropylene non-woven fabric. However, the adhesive tapes made of the raw materials described above all have the following two common disadvantages: (1) having less elasticity and therefore not applicable to the active parts such as joints or the like; (2) the blocking property to liquid is so poor that the tapes can not prevent the affect of foreign liquid on applied parts or the infiltration of internal medicine liquid.

In view of this, there have been some inventions striving to solve parts of the problems described above. For example, Europe Patent EP1097185 introduces a non-woven fabric of a metallocene-catalyzed polyethylene (a copolymer of ethylene and α-olefins) as a substrate material for dressing, plaster and tape or the like. However, generally speaking, polyethylene particles have a high crystallinity and are hard to process, and especially, the metallocene-catalyzed polyethylene can not be used to manufacture non-woven fabrics by a spunbond method. Though a few commercial polyethylene non-woven fabrics can be prepared by a flash-spun method, but because they are too tenacious and lack elasticity, they are hard to extend.

Therefore, at present, it is desirable to develop a medical adhesive tape with easy processability, good elasticity and liquid blocking property, and the medical articles made of the same, such as non-woven fabric adhesive tape.

### DISCLOSURE OF THE INVENTION

An object of the invention is to provide a medical adhesive tape with easy processability, good elasticity and liquid blocking property.

As an aspect of the invention, the invention provides a medical adhesive tape, which comprises:

(1) A non-woven fabric substrate made of a metallocene polypropylene copolymer;

(2) A medical pressure-sensitive adhesive coated on at least one side of said substrate.

As another aspect, the invention provides a medical article, which comprising the medical adhesive tape described above and a medicament.

According to some embodiments of the invention, as compared with the conventional adhesive tapes and articles, the medical adhesive tape of the invention and the articles made of the same have higher elasticity and provide the skin with a more comfortable feeling, and therefore are applicable to bent joints and any active parts; the substrate has water resistance strong enough to prevent the immersion of foreign moisture completely and to reduce the infiltration of the internal liquid (e.g., wound fluid), which is especially effective for short-term blocking.

### DESCRIPTION OF THE DRAWING

FIG. 1 is a schematic view of a non-woven fabric single-side adhesive tape according to an embodiment of the invention.

FIG. 2 is a schematic view (left: sectional view; right: front view) of a non-woven fabric adhesive tape used as a dressing according to an embodiment of the invention.

FIG. 3 is a schematic view (left: sectional view; right: front view) of an elastic water-resistant non-woven fabric adhesive tape used as a plaster.

FIG. 4 is a schematic view (left: sectional view; right: front view) of an elastic water-resistant non-woven fabric adhesive tape used as a woundplast.

The invention will be described in detail below in view of the drawings and examples. It shall be noted that these description and examples are all exemplary embodiments and should not limit the invention in any way. In the specification, unless stated otherwise, all of the contents of the parts, percents, proportions and the like are on the basis of weight.

### SPECIFIC MODE OF CARRYING OUT THIS INVENTION

The invention provides a medical adhesive tape which comprises:

(1) a non-woven fabric substrate made of a metallocene polypropylene copolymer;

(2) a medical pressure-sensitive adhesive coated on at least one side of said substrate.

The invention further provides a medical article which includes the medical adhesive tape described above and a medicament.

The term “article” used in the invention may include dressing, plaster and woundplast and the like, for instance.

According to some embodiments, the non-woven fabric substrate of the metallocene polypropylene copolymer used in the adhesive tape of the invention has the following characteristics: a gram weight of 20-100 g/m²; a thickness of 0.05-0.4 mm; elastic recovery rates in a longitudinal direction and a transverse direction of up to 85% or more; and a hydrostatic pressure value for the non-woven fabric of 25 cm water column or more.

In the invention, the term “metallocene polypropylene copolymer” means a copolymer of propylene and at least one additional α-olefin produced by olefin polymerization using a metallocene catalyst. In the invention, the α-olefins applicable for performing a copolymerization with propylene include but not limited to, ethylene, butylene, amylene, hexene, heptene, octene, nonene, decene, decylene, dodecene. According to some embodiments, the metallocene polypropylene copolymer being applicable to the invention has a weight-average molecular weight of 150,000-250,000, a molecular weight distribution≦2.5, a melt flow rate of 0.5-35 g/10 min, a crystallinity of 3-30%, a tensile strength of 15.2-27.6 MPa, an elongation rate of 100-1,500%, an elastic recovery rate of 80-97%, a T_{g} of −20 to −30° C., and a Tₘ of 40-160° C. According to certain particularly preferred embodiments, propylene units in the metallocene polypropylene copolymer being applicable to the invention have a mole fraction of 65% or more, and more preferably 70% or more.

As to the metallocene-catalyzed polyolefin non-woven fabric, as compared with metallocene polyethylene material, metallocene polypropylene material has the following several advantages: (1) having a better tensile strength and a better wear resistance; (2) the probability of stratification is reduced greatly; and (3) the processing window (e.g. temperature range) is wide.

The metallocene catalyst for olefin polymerization is generally a catalytic system consisted of a metallocene compound as a major catalyst and a Lewis acid as a co-catalyst. The metallocene compound is an organometallic complex compound formed by transition metal atoms in a metallocene cycle coordination. When metallocene is used as an olefin catalyst, it has the following advantages due to its special structure: making the comonomer disperse uniformly; controlling the microcosmic stereochemical structure of polyolefin accurately; able to prepare copolymers with a relatively low crystallinity; and the like. Polyolefin elastomers with good elasticity can be prepared using these characteristics of metallocene catalysts. Metallocene polyolefin elastomers have the characteristics of low price, high elasticity and good compatibility with other materials.

The metallocene catalyst system used for preparing polypropylene elastomers includes bridging metallocene catalyst system, non-bridging metallocene catalyst system, mono-metallocene catalyst system and metallocene/boride catalyst system. For example, the special propylene-ethylene copolymer elastomers of Vistamaxx series provided by ExxonMobil Company are preferably used in some embodiments of the invention wherein the molar fraction of propylene is 65% or more. As to the metallocene polypropylene copolymer elastomers, for example, are also disclosed in U.S. Pat. No. 6,476,172 B1 which describes an elastic fibre of a random copolymer of propylene and α-olefin catalyzed by metallocene and the process thereof, which is hereby incorporated by reference herein in its entirety. As to the metallocene polypropylene copolymer elastomers, for example, it can also be referred to:

Chien J et al (Polymer Chemistry, 1991, 28(11): 1603-1607) synthesized a thermoplastic polypropylene elastomer with a crystalline-amorphous steric segmented structure using rac-anti-Et(Me₄C₅)(Ind)TiCl₂, an equimolar mixture of rac-anti-Et(Me₄C₅)(Ind)TiCl₂/rac-syn-Et(Me₄C₅)(Ind)TiCl₂ and an unsymmetric bridging metallocene catalyst of rac-anti-Et(Me₄C₅)(Ind)TiMe₂;

Gauthier W J et al (Macromolecules, 1995, 28(11): 3779-3786) synthesized a polypropylene elastomer using metallocene catalysts of Me₂C(Cp)(Ind)TiCl₂, Me₂C(Cp)(Ind)ZrCl₂, Me₂C(Cp)(Ind)HfCl₂, Me₂Si(Cp)(Ind)ZrCl₂ and Me₂Si(Cp)(Ind)HfCl₂, wherein Cp is a cyclopentadienyl;

Resconi L et al (Chemical Reviews, 2000, 100(4): 1253-1345) synthesized a completely amorphous polypropylene elastomer with a weight-average molecular weight of up to 100,000 to 400,000 and good elasticity and optical performance using a symmetric achiral bridging metallocene catalyst of Me₂Si(Flu)₂ ZrCl₂Me₂Si(Flu)₂ZrMe₂C₂ᵥ;

Coates G (Chemical Reviews, 2000, 100(4): 1223-1252) synthesized a polypropylene elastomer using bridge vibration-free metallocene catalysts of (2-PhInd)₂ZrCl₂, (2-PhInd)₂HfCl₂, (2-PhInd)₂Zr(CH₂Ph)₂ and (2-PhInd)₂Hf(CH₂Ph)₂;

Collete J W et al (Macromolecules, 1989, 22(10): 3851-3858) synthesized a polypropylene elastomer using a MR₄ metallocene catalyst comprising Al₂O₃ as a carrier, wherein M is titanium, zirconium or hafnium; R is CH₂Ph, Me₃CH₂ or PhCMe₂CH₂;

Tullock C W et al (Journal of Polymer Science. Part A. Polymer Chemistry, 1989, 27(9): 3063-3081) synthesized a steric segmented polypropylene elastomer using a MAr₂ [for example, Ti(C₆H₅CH₃)₂] metallocene catalyst comprising Al₂O₃ as a carrier.

Metallocene polypropylene copolymer can be made into a non-woven fabric easily using known non-woven fabric producing technologies. The non-woven fabrics in preferred embodiments for example include a Marnix elastic non-woven fabric produced by a spunbond method (National Bridge Industrial Co. Ltd., Shenzhen, China). Marnix is a novel non-woven fabric product manufactured using a spunbond process taking a special elastomer Vistamaxx (a metallocene propylene-ethylene copolymer produced by ExxonMobil Chemical Company) as a main raw material. It combines the advantages of non-woven fabrics and elastic fabrics and has soft feeling as well as elasticity. It has an elongation rate of up to 200-500% and an elastic recovery rate of 90% or more. Vistamaxx has a molecular weight of 150,000-250,000, a molecular weight distribution≦2.5, a melt flow rate of 0.5-35 g/10 min, a crystallinity of 3-30%, a tensile strength of 15.2-27.6 MPa, an elongation rate of 100-1,500%, an elastic recovery rate of 80-97%, a T_{g} of −20 to −30° C., and a Tₘ of 40-160° C. A mole fraction of propylene units in Vistamaxx is 65% or more. A hydrostatic pressure value thereof is up to 25-70 cm which may vary in dependence on the specific thicknesses of the non-woven fabric. Generally, the thicker the non-woven fabric is, the higher the hydrostatic pressure value will be.

According to some embodiments, the medical pressure-sensitive adhesive used in the adhesive tape of the invention is an acrylic ester or a rubber based pressure-sensitive adhesive. According to some embodiments, the thickness of a coated dry glue is 10-100 g/m².

According to some embodiments, the adhesive force of the pressure-sensitive adhesive useful in the invention meets the following test: when a polyethylene terapthalate film with a thickness of 25 microns is used as a substrate coated with the adhesive to test the adhesive force thereof to a steel plate, the adhesive force of this pressure-sensitive adhesive should be above 0.1 N/mm. This is because the non-woven fabric used in the invention has a water resistance and the water resistance increases with the increment of the gram weight, that is, the increment of thickness of the non-woven fabric. Therefore, an adhesive tape can be prepared by recombining a pressure-sensitive adhesive and the surface of a non-woven fabric well only if the pressure-sensitive adhesive has a minimal steel plate peeling force of above 0.1 N/mm. Additionally, under a precondition of the same coating basis weight, the degrees from easy to difficult for the recombination on the surface of the non-woven fabric are in the following sequence: solvent type synthetic rubber adhesive>solvent type acrylic ester adhesive>aqueous acrylic ester adhesive.

According to some embodiments, before being used, the metallocene polypropylene non-woven fabric can be subjected to a surface treatment so as to change the surface performance on the fabric, for example, to improve wettability to increase the interface binding performance with an adhesive. The known surface treatment methods can all be applied to the invention. Examples include, but not limited to, corona treatment, plasma treatment, flame treatment and the like.

The invention can be implemented by the following process:

A pressure-sensitive adhesive is coated on a release paper and is put into an oven to be dried. Under assistance of rollers, the pressure sensitive adhesive was recombined with a non-woven fabric substrate and the glue layer was transferred to the non-woven fabric.

Besides producing adhesive tapes, the invention can also be used to produce the products of dressing, plaster (e.g., a traditional Chinese medicine plaster) and woundplast (e.g., first aid bandage or dressing) or the like. However, it should be understood that the invention can also be applied to the situations demanding elastic water-resistant adhesive tapes in industry, for example, be used as industrial adhesive tapes applicable to electronic and telecommunication markets.

In this invention, a medical adhesive tape is prepared first using a metallocene polypropylene copolymer non-woven fabric as a tape substrate and a medical grade pressure-sensitive adhesive. The medical adhesive tape made of this base material and the medical article made of the tape both have the advantages of elasticity, water resistance and comfortable touch to skin simultaneously. Additionally, the processing process is simple and the products attach to the skin of human even more and have a wider application range. When the medical article according to the invention such as adhesive tape is applied to active joints, it can be attached to the active parts completely without tension and the medicine liquid will not penetrate the tape to contaminate the clothes of users/wearers of the tape. Furthermore, users can take a shower normally without affecting the action of the medicine on the plaster.

### Examples

### Testing Methods

### Testing Method for Elastic Recovery Rate

Following FZ/T 70006-2004 Standard, a multipurpose electronic tensiometer (Instron5569, instron Company, Shanghai, China) is utilized. The operation is as follows: (1) a 2.54 cm×10 cm sample is fixed on a clamp of the tensiometer; (2) a force of 1 N is applied to the sample and the length of the sample at the time is recorded to be L₀; (3) operating the tensiometer to draw the sample to a fixed elongation (an elongation of 20% in this invention) and then keeping fort min, and the length of the sample at the time is recorded to be L₀₁; (4) the tensile force is withdrawn and the sample recovers to a natural state and keeps for 3 min; (5) a tensile force of 1 N is again applied to the sample and the length of the sample at the time is recorded to be L₀′,

The measured elastic recovery rate=(L₀₁−L₀′)/(L₀₁−L₀)×100%

### Testing Method for Hydrostatic Pressure

Following ISO811:1981 1 Standard and using a YG825 type fabric hydrostatic pressure tester (Ningbo Textile Instrument Factory, Ningbo, China) with an increasing speed for water pressure of 60 cm/min and a water temperature of 20° C. (standard atmosphere pressure). The operation is as follows: a sample with an area of above 10 cm×10 cm is cut and clamped on the tester according to the standard (as to the adhesive tape, it was clamped with the glue face downward); (2) an increasing speed for water pressure of 60 cm/min is selected; (3) The START button is pressed and the water pressure starts to increase stably, and when the third water drop begins to emerge or the third infiltration position begins to appear on the surface of the non-woven fabric, the pressure is recorded and regarded as the hydrostatic pressure.

### Example 1

### Adhesive Tape

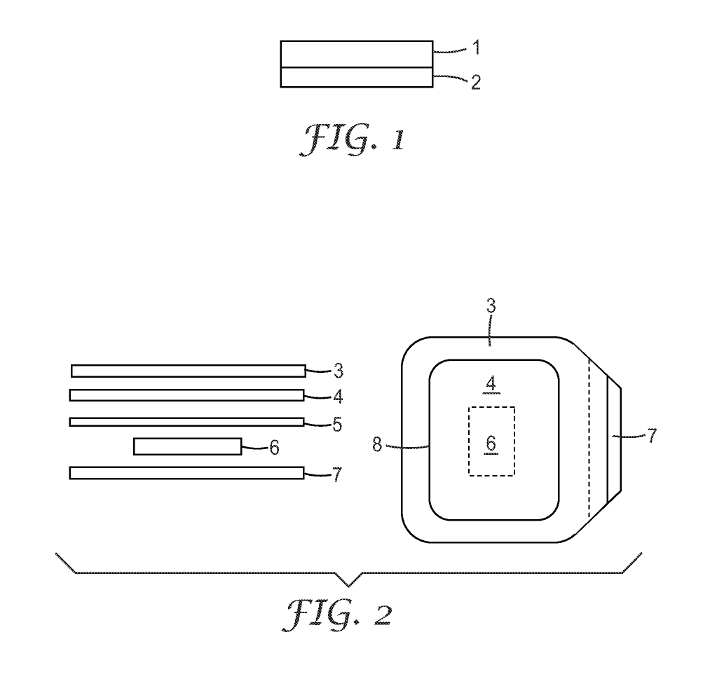

A PS-57E aqueous acrylic ester adhesive (Xi'an Tianyun Industrial Co. Ltd., Xian, China) with a dry glue basis weight of 30.0 g/m² was coated on a single-side release paper (Baoyan Industrial Technical Co. Ltd., Shanghai, China) with a basis weight of 120 g/m² and was put into an oven to be dried. Under an effect of milling rollers, it was combined with a Marnix elastic non-woven fabric (National Bridge Industrial Co. Ltd., Shenzhen, China) with a basis weight of 20 g/m² and a thickness of 60 μm and the adhesive layer was transferred to the non-woven fabric. The obtained non-woven fabric tape has a peeling force for steel plate of 0.311 N/mm, a hydrostatic pressure of 31.2 cm and elastic recovery rates in a longitudinal direction and a transverse direction of 95.33% and 92.38%, respectively. The prepared tape was shown in FIG. 1. The adhesive tape was a double-layer structure which included from the top down: a layer of metallocene polypropylene copolymer non-woven fabric 1; and a layer of medical pressure-sensitive adhesive 2. When applied to elbow joints or knee joints, the obtained adhesive tape had excellent conformability and comfortability, as well as no warping. Additionally, the normal action of the joint parts was not affected because there was no tension feeling of the known non-woven fabric adhesive tapes. This adhesive tape stuck normally for 7 days or more continuously. Daily showers were carried out during the process of application (about 30 min each time).

### Example 2

### Adhesive Tape

A PS-58E aqueous acrylic ester adhesive (Xi'an Tianyun Industrial Co. Ltd., Xian, China) with a dry glue basis weight of 30.0 g/m² was coated on a single-side release paper (Baoyan Industrial Technical Co. Ltd., Shanghai, China) with a basis weight of 120 g/m² and was put into an oven to be dried. Under an effect of milling rollers, it was combined with a Marnix elastic non-woven fabric (National Bridge Industrial Co. Ltd., Shenzhen, China) with a basis weight of 40 g/m² and a thickness of 117 μm and the adhesive layer was transferred to the non-woven fabric. The obtained non-woven fabric adhesive tape had a peeling force to steel plate of 0.462 N/mm, a hydrostatic pressure of 38.0 cm and elastic recovery rates in a longitudinal direction and a transverse direction of 94.13% and 91.68%, respectively.

### Example 3

### Adhesive Tape

An acrylic ester adhesive (an isooctyl acrylate-acrylic acid copolymer adhesive of 95.5/4.5 (a weight ratio) prepared according to U.S. Pat. No. RE24960 (Ulrich)) with a dry glue basis weight of 43.8 g/m² was coated on a single-side release paper (Baoyan Industrial Technical Co. Ltd., Shanghai, China) with a basis weight of 120 g/m² and was put into an oven to be dried. Under an effect of milling rollers, it was combined with a Marnix elastic non-woven fabric (National Bridge Industrial Co. Ltd., Shenzhen, China) with a basis weight of 60 g/m² and a thickness of 180 μm and the adhesive layer was transferred to the non-woven fabric. The obtained non-woven fabric adhesive tape had a peeling force for steel plate of 0.240 N/mm, a hydrostatic pressure of 57.0 cm and elastic recovery rates in a longitudinal direction and a transverse direction of 93.55% and 90.82%, respectively.

### Example 4

### Adhesive Tape

A synthetic rubber adhesive (containing about 25% of a polystyrene-isoprene-styrene copolymer (Kraton 1117(Kraton LLC, Houston, Tex.)), about 25% of an aromatic modified C5 resin adhesion-promoting agent, about 49% of a toluene-heptane mixed solvent and about 0.1% of an antioxidant) with a dry glue basis weight of 38.4 g/m² was coated on a single-side release paper (Baoyan Industrial Technical Co. Ltd., Shanghai, China) with a basis weight of 120 g/m² and was put into an oven to be dried. Under an effect of milling rollers, it was combined with a Marnix elastic non-woven fabric (National Bridge Industrial Co. Ltd., Shenzhen, China) with a basis weight of 80 g/m² and a thickness of 245 μm and the adhesive layer was transferred to the non-woven fabric. The obtained non-woven fabric adhesive tape had a peeling force for steel plate of 1.141 N/mm, a hydrostatic pressure of 64.5 cm and elastic recovery rates in a longitudinal direction and a transverse direction of 93.00% and 88.02%, respectively.

### Example 5

### Adhesive Tape

A synthetic rubber adhesive (containing about 25% of a polystyrene-isoprene-styrene copolymer (Kraton 1117(Kraton LLC, Houston, Tex.)), about 25% of an aromatic modified C5 resin adhesion-promoting agent, about 49% of a toluene-heptane mixed solvent and about 0.1% of an antioxidant) with a dry glue basis weight of 49.8 g/m² was coated on a single-side release paper (Baoyan Industrial Technical Co. Ltd., Shanghai, China) with a basis weight of 120 g/m² and was put into an oven to be dried. Under an effect of milling rollers, it was combined with a Marnix elastic non-woven fabric (National Bridge Industrial Co. Ltd., Shenzhen, China) with a basis weight of 100 g/m² and a thickness of 308 μm and the adhesive layer is transferred to the non-woven fabric. The obtained non-woven fabric adhesive tape had a peeling force for steel plate of 1.481 N/mm, a hydrostatic pressure of 68.9 cm and elastic recovery rates in a longitudinal direction and a transverse direction of 91.00% and 87.67%, respectively.

### Example 6

### A Dressing Based on the Adhesive Tape

The materials used in the example were as follows:

frame paper (a basis weight of 125 g/m², Naiheng Paper Co. Ltd., Guangzhou, China);

elastic non-woven fabric, adhesive, cotton pad (Huilong Hygienic Material Co. Ltd., Shanghai, China); and

release paper (a basis weight of 100 g/m², Baoyan Industrial Technical Co. Ltd., Shanghai, China).

The dressing was prepared using an elastic water-resistant non-woven fabric tape shown in FIG. 2 (left: sectional view; right: front view). This dressing structure included a five-layer structure from the top down: a layer of frame paper 3(including cutting line 8); a layer of 40 g/m² elastic non-woven fabric 4; a layer of 30 g/m² PS-57E aqueous acrylic ester adhesive 5; a layer of cotton pad 6; and a layer of 100 g/m² single-side release paper 7.

The processing process was as follows: (1) a non-woven fabric adhesive tape was prepared by coating; (2) the obtained adhesive tape was put onto a unreeling roller of a die cutting machine; (3) in a rate of 4 m/min, a frame paper with a die cut shape was combined together with the surface of the non-woven fabric by a hot-roller at 200° C. and the primary release paper was peeled off; (4) The adhesive face was added with a cotton pad and was attached with a product release paper; (5) the finished product was die cut into a shape.

The obtained dressing had a release force for frame paper of 3.2 g/mm, a release force for adhesive face of 1.2 g/mm and the peeling force of the adhesive face to the steel plate was 0.372 N/mm.

### Example 7

### A Plaster Based on the Adhesive Tape

The materials used in the example were as follows:

elastic non-woven fabric, adhesive, cotton pad (Huilong Hygienic Material Co. Ltd., Shanghai, China); and

release paper (a basis weight of 130 g/m², Baoyan Industrial Technical Co. Ltd., Shanghai, China).

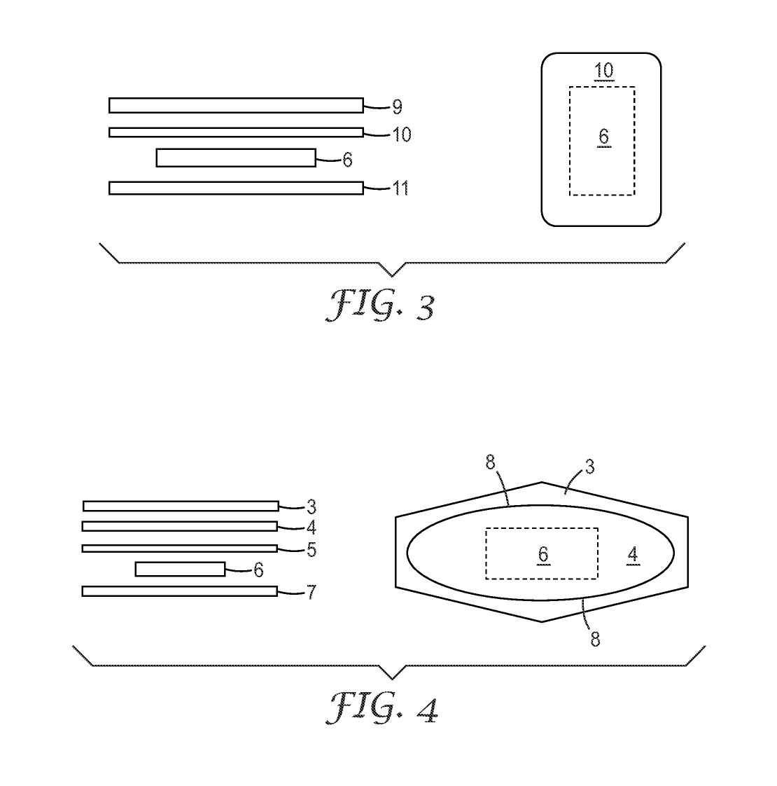

The plaster prepared using an elastic water-resistant non-woven fabric tape was shown in FIG. 3 (left: sectional view; right: front view).

This plaster structure included a four-layer structure from the top down: a layer of 80 g/m² elastic non-woven fabric 9; a layer of 30 g/m² AK2247 synthetic rubber adhesive 10; a layer of cotton pad 6; and a layer of 130 g/m² single-side release paper 11.

The processing process was as follows: (1) a non-woven fabric adhesive tape was prepared by coating; (2) the obtained tape was put onto a unreeling roller of a die cutting machine; (3) the primary release paper was peeled off; (4) The adhesive face was added with a cotton pad and was attached with a product release paper; (5) the finished product was die cut into a shape.

The obtained plaster had a release force of 0.7 g/mm and the peeling force of the adhesive face to the steel plate was 1.110 N/mm.

### Example 8

### A Woundplast Based on the Adhesive Tape

The materials used in the example were as follows:

frame paper (a basis weight of 125 g/m², Naiheng Paper Co. Ltd., Guangzhou, China);

elastic non-woven fabric, adhesive, cotton pad (Huilong Hygienic Material Co. Ltd., Shanghai, China); and

release paper (a basis weight of 100 g/m², Baoyan Industrial Technical Co. Ltd., Shanghai, China).

The woundplast prepared using an elastic water-resistant non-woven fabric tape was shown in FIG. 4 (left: sectional view; right: front view). This woundplast structure included a five-layer structure from the top down: a layer of frame paper 3(including cutting line 8); a layer of 40 g/m² elastic non-woven fabric 4; a layer of 30 g/m² PS-57E aqueous acrylic ester adhesive 5; a layer of cotton pad 6; and a layer of 100 g/m² single-side release paper 7. The processing process was as follows: (1) a non-woven fabric tape was prepared by coating; (2) the obtained tape was put onto a unreeling roller of a die cutting machine; (3) in a rate of 4 m/min, a frame paper with a die cut shape was combined together with the surface of the non-woven fabric by a hot-roller at 200° C. and the primary release paper was peeled off; (4) The adhesive face was added with a cotton pad and was attached with a product release paper; (5) the finished product was die cut into a shape.

The obtained woundplast has a release force for frame paper of 3.0 g/mm, a release force for adhesive face of 1.1 g/mm and the peeling force of the adhesive face to the steel plate was 0.301 N/mm.

1.-9. (canceled)

10. A medical adhesive tape which includes:
(a) a non-woven fabric substrate made of a metallocene polypropylene copolymer;
(b) a medical pressure-sensitive adhesive coated on at least one side of said substrate.

11. The medical adhesive tape of claim 1, wherein the non-woven fabric substrate has the a basis weight of 20-100 g/m², a thickness of 0.05-0.4 mm, and a hydrostatic pressure value for the non-woven fabric of 25 cm water column or more.

12. The medical adhesive tape of claim 2, wherein the non-woven fabric substrate has elastic recovery percentages in a longitudinal direction and a transverse direction of up to 85% or more.

13. The medical adhesive tape of claim 1, wherein the polypropylene has a weight-average molecular weight of 150,000-250,000, a molecular weight distribution of 2.5 or less, a melt flow rate of 0.5-35 g/10 min, a crystallinity of 3-30%, a tensile strength of 15.2-27.6 MPa, an elongation of 100-1,500%, an elastic recovery rate of 80-97%, a T_{g} of −20 to −30° C. and a Tₘ of 40-160° C.

14. The medical adhesive tape of claim 1, wherein a mole fraction of propylene units in the polypropylene is 65% or more.

15. The medical adhesive tape of claim 1, wherein the polypropylene non-woven fabric is a non-woven fabric prepared by a spunbond method.

16. The medical adhesive tape of claim 1, wherein the polypropylene non-woven fabric is treated by a surface-treating method selected from corona treatment, plasma treatment and flame treatment.

17. A medical article tape of claim 1, further comprising a medicament.
